SEQUENCE LISTING (3 sequences)

SEQ ID NO: 1 - protein, 497 aa, Artificial sequence
  Synthetic peptide
MASIIMEVATMQAKLTKKEFIEWLKTSEGKQFNVDLWYGFQCFDYANAGWKVLFGHTLKGLGAKDIPFANNFDGLATVYQNTPDFLAQPGDMVVFGSNYGAGYGHVAWVIEATLDYIIVYEQNWLGGGWTDRIEQPGWGWEKVTRRQHAYDFPMWFIRPNFKSATAPASIQSPTQASKKETAKPQPKAVELKIIKDVVKGHDLPKRGGNPKGIVIHNDAGSKGATAEAYRNGLVNAPSSRLEAGIAHSYVSGNTVWQALDESQVGWHTANQLGNKYYYGIEVCQSMGADNATFLKNEQATFQECARLLKKWGLPANRNTIRLHNEFTSTSCPHRSSVLHTGFDPVTRGLLPEDKRLQLKDYFIKQIRAYMDGKIPVATVSNESSASSNTVKPVAELMPPVPAGYTLDKNNVPYKKEQGNYTVANVKGNNVRDGYSTNSRITGVLPNNTTITYDGAYCINGYRWITYIANSGQRRYIATGEVDIAGNRISSFGKFSAV

SEQ ID NO: 2 - protein, 101 aa, unknown
  Prophage SA2USA
MPPVPAGYTLDKNNVPYKKEQGNYTVANVKGNNVRDGYSTNSRITGVLPNNTTITYDGAYCINGYRWITYIANSGQRRYIATGEVDIAGNRISSFGKFSAV

SEQ ID NO: 3 - protein, 96 aa, unknown
  Pitti20
MKRKKPKGWSENPYGTYYKKVDKTFIVGSEKIETRIGSPFLSAPSGGHVTPNQKMTFDYLAQQDGYEWGQLENNRGQQEFVPIRPLSQKEYWGILK

(19) United States
(12) Patent Application Publication
(10) Pub. No.: US 2009/0304673 A1
(43) Pub. Date: Dec. 10, 2009

(54) Method of Treatment of Inflammatory Diseases

(76) Inventors: Bernd Buchberger, Zeitlarn (DE); Holger Grallert, Regensburg (DE); Ingrid Wanninger, Regensburg (DE)

Correspondence Address: FULBRIGHT & JAWORSKI L.L.P., 600 CONGRESS AVE., SUITE 2400, AUSTIN, TX, 78701, US

(21) Appl. No.: 12/482,174

(22) Filed: Jun. 10, 2009

Related U.S. Application Data
(60) Provisional application No. 61/060,314, filed on Jun. 10, 2008.

(51) Int. Cl. A61K 38/48; A61K 38/43; C12N 9/00

(52) U.S. Cl. 424/94.67; 424/94.1; 435/183

(57) ABSTRACT

The present invention relates to a method of treatment of an inflammatory disease, in particular sepsis, in particular sepsis caused by gram-positive bacteria, wherein the method comprises the step of administering to a subject in need thereof a sufficient amount of bacterial cell wall degrading enzymes. The present invention relates further to pharmaceutical compositions comprising bacterial cell wall degrading enzymes.

### CROSS-REFERENCE TO RELATED APPLICATION

This application claims benefit of U.S. Provisional Application Ser. No. 61/060,314, filed Jun. 10, 2008, the entire contents of which are incorporated by reference.

### BACKGROUND

A. Field of the Invention

The present invention relates to a method of treatment of an inflammatory disease, in particular sepsis, in particular sepsis caused by gram-positive bacteria, wherein the method comprises the step of administering to a subject in need thereof a sufficient amount of bacterial cell wall degrading enzymes. The present invention relates further to pharmaceutical compositions comprising bacterial cell wall degrading enzymes.

B. Background of the Invention

Sepsis is a complex, multifactoral and rapidly progressing disease characterized by an excessive inflammatory response to infection that leads to organ failure and death. Severe forms of sepsis such as septic shock result in up to 80% mortality. In the US, sepsis is the 10th leading cause of death, killing over 200,000 people annually.

Sepsis is often confused with blood poisoning, a condition also associated with the presence of bacteria in a patient's blood. Sepsis is a complex illness consisting in the overreaction of the body to an infection. Central to the pathogenesis of sepsis is the host immune response, which triggers a systemic chain reaction that leads to the activation of the inflammatory response and the coagulation cascade, ultimately leading to multiple organ damage, organ failure, and eventually death. The high mortality associated with sepsis is partly a consequence of its rapid progression of the disease, with patients often progressing from sepsis to severe sepsis, septic shock and organ damage in a matter of hours. Despite significant progress in the management of sepsis, mortality remains unacceptably high, estimated at 20-50% for severe sepsis and 45-80% for septic shock (EMEA, 2005). In the US, sepsis causes more than 200,000 deaths per year, more than some common forms of malignancies including lung, colon and breast cancer (www.survivingsepsis.org).

Up to 70% of sepsis cases are caused by bacterial infection, with the remainder of cases due to fungi, viruses and parasites, although polymicrobial infections can also be present. Recent epidemiological studies suggest that there might be a similar contribution of gram-negative and gram-positive infections, with the recent increase in gram-positive infections likely due be a consequence of the increasing emergence of drug-resistant bacterial strains, for example methicillin-resistant Staphylococcus aureus (MRSA). Almost half of all infections leading to sepsis originate in the lung, followed by infections of the bloodstream, the urinary tract and the abdomen. The body's overreaction to the infection or toxins released by the pathogen then leads to a widespread inflammation causing extensive collateral damage to the host's microcirculation, increased blood clotting, and impaired fibrinolysis, culminating in the formation of small blood clots that eventually damage vital organs. Individuals with a weakened immune system are at an increased risk of sepsis, in particular the elderly, children, and immunocompromised patients, including individuals infected with HIV, cancer and transplant patients receiving immunosuppressive therapy. One of the key risk factors is the presence of foreign objects in patients undergoing surgery, for example intravenous lines and urinary catheters.

In the US, severe sepsis has been estimated to affect over 750,000 individuals per year, equating to 300 cases per 100,000 population. Importantly, surveillance in the US has shown that the incidence of severe sepsis has doubled in the past 20 years and is expected to increase by 1.5% per annum, such that the total number of cases might rise to one million by 2010 (www.emedicine.com, 2003). Although European studies have estimated the sepsis incidence at 51-115 cases per 100,000 population, experts agree that there is no reason for the incidence in these two geographical areas to differ.

The rise in sepsis is following advances in medical practice, which have led to the extension of the normal lifespan, the increase in the number of invasive surgical procedures, and the size of the patient pool with weakened immune systems:
the aging of the population has led to an increase in the number of people with a weaker immune system or suffering from chronic conditions that increase the likelihood of developing sepsis, such as diabetes, cancer, blood diseases, and liver, kidney and lung infections;
the number of immunosuppressed patients is increasing, including cancer, HIV-infected, and transplant patients;
the overuse of antibiotics for infections not caused by bacteria have led to high levels of resistant bacteria, rendering the treatment of sepsis very difficult.

For many years the terms sepsis, septicemia, bacteremia, infection, septic shock and toxic shock were used interchangeably. However, with time it became clear that the reason for the ultimate multiple-organ failures was systemic inflammation, rather than the original infection itself. In order to eliminate the prevailing climate of confusion and ambiguity, in 1992 the American College of Chest Physicians and the Society of Critical Care Medicine organized a consensus conference aimed at establishing a set of definitions that could be applied to patients with sepsis and its sequelae (ACCP-SCCM Consensus Conference, 1992). According to these definitions, sepsis is a progressive disease that consists of various stages, which can be clearly differentiated based on specific physiological occurrences.

The consensus meeting established the following definitions for the different stages of the disease:
systemic inflammatory response syndrome (SIRS) is a widespread inflammatory response to any of a wide variety of severe clinical insults. Visible symptoms resulting from these conditions are: fever, chills, severe shaking, tachycardia, confusion, disorientation and agitation, rash on the skin and pain in the joints;
sepsis is defined as the systemic inflammatory response to infection. Sepsis patients thus represent a subset of SIRS patients;
severe sepsis is associated with organ dysfunction, hypoperfusion or hypotension. Clinical manifestations of hypoperfusion may include lactic acidosis, oliguria or an acute alteration in mental status. Sepsis can turn into severe sepsis within a few hours;
septic shock, a subset of severe sepsis, is defined as sepsis-induced hypotension despite adequate fluid resuscitation, combined with perfusion abnormalities that may include, but are not limited to, lactic acidosis, oliguira or an acute alteration in mental status (Patients receiving inotropic or vasopressor agents may no longer be hypotensive by the time they manifest hypoperfusion abnormalities or organ dysfunction; yet they would still be considered to have septic shock. These patients generally present three cardiovascular upsets: vasodilation, reduced stroke volume, and microcirculatory failure. Importantly, over half of all patients with septic shock have bacteremia. Although the exact sequence of the events that lead to septic shock is not fully understood, two key factors associated with septic shock are the predominance of the pro-inflammatory over the anti-inflammatory response, and coagulation over fibrinolysis);
multiple organ dysfunction syndrome (MODS) is characterized by the presence of altered organ function in an acutely-ill patient such that homeostasis cannot be maintained without intervention;
multi-organ failure: the activation of the coagulation cascade appears to be an essential component in the development of multi-organ failure. Organs most commonly affected by sepsis are the brain, heart, lungs, kidneys and liver.

Sepsis most often starts with a localized infection caused by bacteria, fungi, viruses or parasites introduced by insults such as trauma, burn wounds, ischemia-reperfusion and major surgery including cardiopulmonary bypass and abdominal surgery. 40% of infections were due to grampositive infections, while 38% were due to gram-negative infections (Vincent et al., 2006).

The management of sepsis currently relies on: Control of the infection with antibiotic treatment and source control in combination with hemodynamic stabilization by fluid administration, administering vasopressor agents and inotropic agents further in combination with modulation of the sepsis response by administering recombinant protein C and steroids.

Early and appropriate administration of anti-infectives, predominantly intravenous (IV) antibiotics, remains the cornerstone of sepsis therapy. In addition, it is often necessary to eliminate the focus of the infection by surgical excision/removal. This affects predominantly patients with a perforated viscus, ruptured esophagus or diaphragm, severe burns or abscesses, and gangrene. The choice of the antibiotic to be used varies from patient to patient, being selected based on the microorganisms most likely to have caused infection at the suspected site, the potential risk of antibiotic resistance, and known patterns of microorganism presence in the specific community and the hospital.

Empirical therapy is initiated with a broad-spectrum antibiotic that covers gramnegative, gram-positive and anaerobic organisms, until the results of the blood test or culture become available. Following the identification of the pathogen, therapy is switched to a narrow-spectrum agent, a process known as de-escalation:
Antibacterial de-escalation is an approach that attempts to balance the need to provide appropriate initial antibacterial treatment, while limiting the emergence of antibacterial resistance. The goal is that the initial antibacterial regimen will cover the most likely bacterial pathogens while minimizing the chance of resistance. The risk for the latter is reduced by narrowing the scope of the antibacterial regimen as soon as the pathogens and their susceptibility profiles are determined, and by employing the shortest course of therapy clinically acceptable. Antibiotics need to be administered intravenously (IV) at doses high enough to achieve bactericidal serum levels.

However, sepsis is as much a result of an imbalance in the coagulation and fibrinolysis cascades as an overwhelming inflammatory response. Thus, there is a need for new therapeutic substances for the treatment of inflammatory diseases like sepsis.

### SUMMARY OF THE INVENTION

The present invention relates to a method of treatment of an inflammatory disease, in particular sepsis, in particular sepsis caused by gram-positive bacteria, wherein the method comprises the step of administering to a subject in need thereof a sufficient amount of bacterial cell wall degrading enzymes. The present invention relates further to pharmaceutical compositions comprising bacterial cell wall degrading enzymes.

One aspect of the invention concerns a method of treating an inflammatory disease in a subject (e.g., humans, mammals, animals, etc.) comprising administering an efficient or effective amount of bacterial cell wall degrading enzymes to said subject. The bacterial cell wall degrading enzyme can be an endolysin, autolysin, lysostaphin, mytanolysin, lytic enzyme similar in function and/or structure to lysostaphin or murolytic enzyme. The bacterial cell wall degrading enzyme can be administered in combination with conventional antibacterial agents (e.g., antibiotics). The bacterial cell wall degrading enzyme can be administered in combination with conventional antibacterial agents (e.g., antibiotics and other endolysins, mytanolysins, autolysins, lysostaphins, lytic enzymes similar to lysostaphin or murolytic enzymes).

Another aspect of the invention concerns a pharmaceutical composition comprising a bacterial cell wall degrading enzyme. The bacterial cell wall degrading enzyme can be an endolysin, autolysin, lysostaphin, mytanolysin, lytic enzyme similar in structure and/or function to lysostaphin or murolytic enzyme. The pharmaceutical composition can further include a conventional antibacterial agent (e.g., an antibiotic agent). The pharmaceutical composition can include different bacterial cell wall degrading enzymes. The pharmaceutical composition can include conventional antibacterial agents, such as antibiotics and other endolysins, autolysins, lysostaphins, mytanolysins, lytic enzymes similar to lysostaphin or murolytic enzymes. The pharmaceutical composition can include a pharmaceutical acceptable a carrier.

The pharmaceutical compositions and methods for their use can “comprise,” “consist essentially of,” or “consist of” any of the ingredients disclosed throughout the specification.

It is contemplated that any embodiment discussed in this specification can be implemented with respect to any method or composition of the invention, and vice versa. Furthermore, pharmaceutical compositions of the invention can be used to achieve methods of the invention.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—A schematic representation of the time dependence of S. aureus distribution in blood with or without treatment of an endolysin as depicted in SEQ ID NO:1. Animals were-infected with 6.5×10⁷ S. aureus cells (black bar=cell number of inocculum). One minute later, they were treated either with the endolysin as depicted in SEQ ID NO:1 or storage buffer (control). Blood of the animals were taken 5, 60 and 360 minutes after infection and dilution series of the blood samples were plated onto LB agar plate. After overnight growth at 30° C., the number of colonies (cfu) were determined.

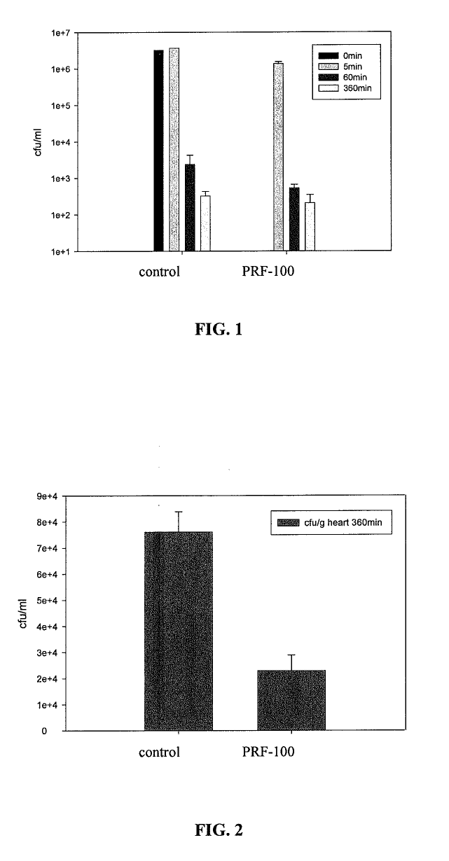

FIG. 2—A schematic representation of the appearance of S. aureus within heart tissue 6 hours post S. aureus infection with or without treatment of an endolysin as depicted in SEQ ID NO:1. Rats were intravenously infected with 6.5×107 S. aureus cells per animal and treated 1 minute later either with the endolysin as depicted in SEQ ID NO:1 or storage buffer. After 6 hours the hearts of the animals were prepared, homogenised and dilution series of the homogenisate were plated onto LB agar plates. After over night cultivation at 30° C., cell forming units were determined.

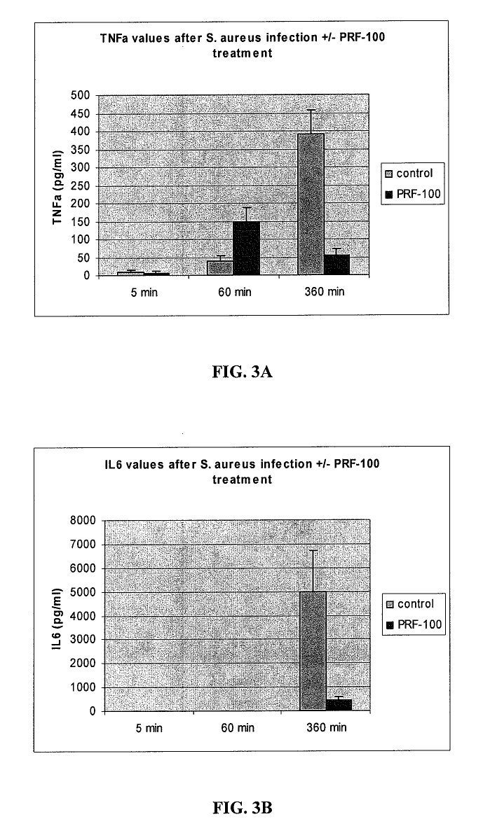

FIG. 3A—A schematic representation of the time dependence of TNF-α levels post S. aureus infection with or without treatment of an endolysin as depicted in SEQ ID NO:1. The mean values and standard deviation of the TNF-α concentration in sera of rats 5, 60 and 360 minutes after infection with 6.5×10⁷ S. aureus cells are blotted (light grey). The TNF-α concentrations after infection and subsequent treatment with the endolysin as depicted in SEQ ID NO:1 are blotted in dark grey.

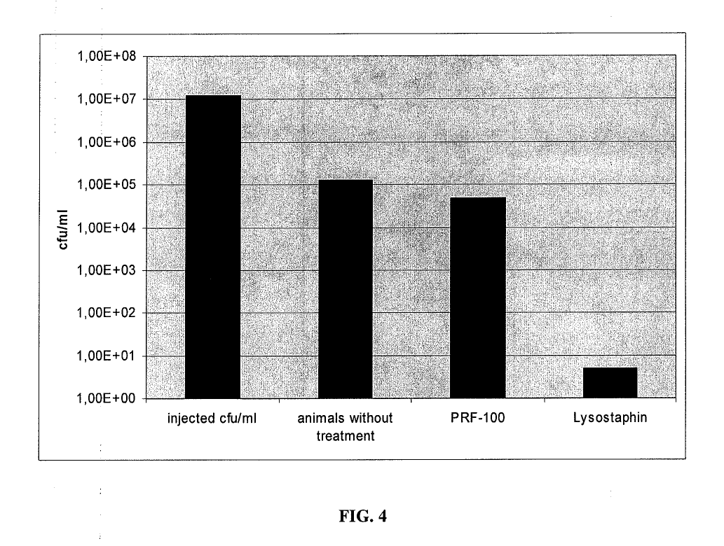

FIG. 3B—A schematic representation of the time dependence of IL-6 levels after post S. aureus infection with or without treatment of an endolysin as depicted in SEQ ID NO:1. The mean values and standard deviation of the IL-6 concentration in sera of rats 5, 60 and 360 minutes after infection with 6.5×10⁷ S. aureus cells are blotted (light grey). The IL-6 concentrations after infection and subsequent treatment with the endolysin as depicted in SEQ ID NO:1 are blotted in dark grey.

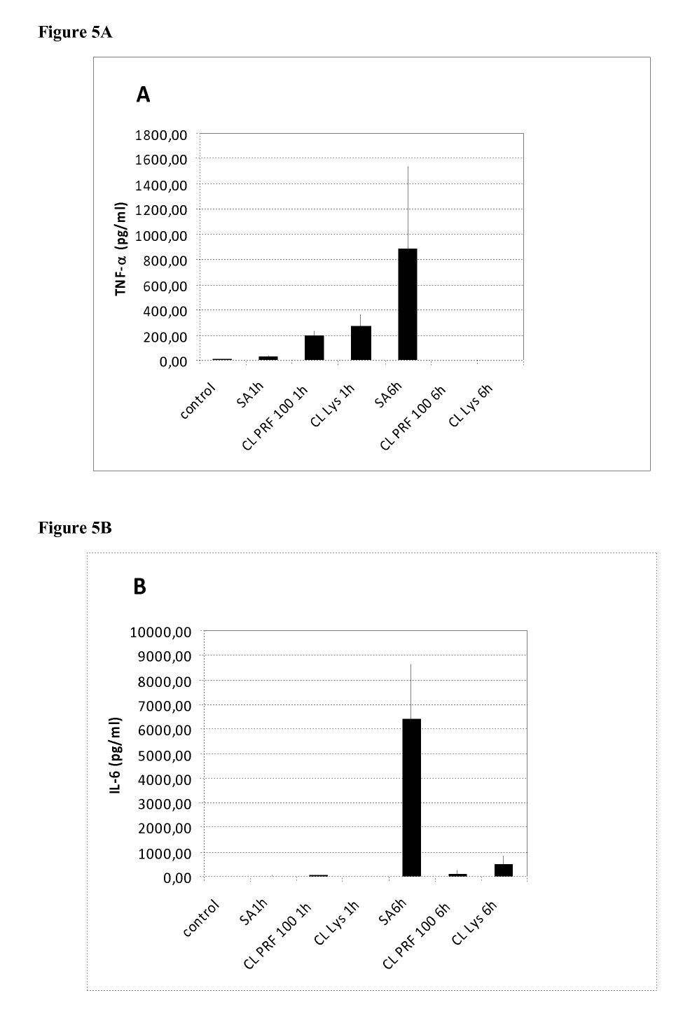

FIG. 4—A schematic representation of S. aureus distribution in blood of animals with or without pre-administration of endolysin PRF-100 as depicted in SEQ ID NO:1 and Lysostaphin. Animals were pretreated with endolysin (PRF-100; 16 mg/kg) or Lysostaphin (8 mg/kg) 15 minutes prior to infection with 1.0×10⁷ S. aureus cells. Blood of animals were taken 10 minutes after infection with S. aureus cells and dilution series of blood were plated onto LB agar plates. After overnight growth at 30° C., the number of colonies (cfu) were determined.

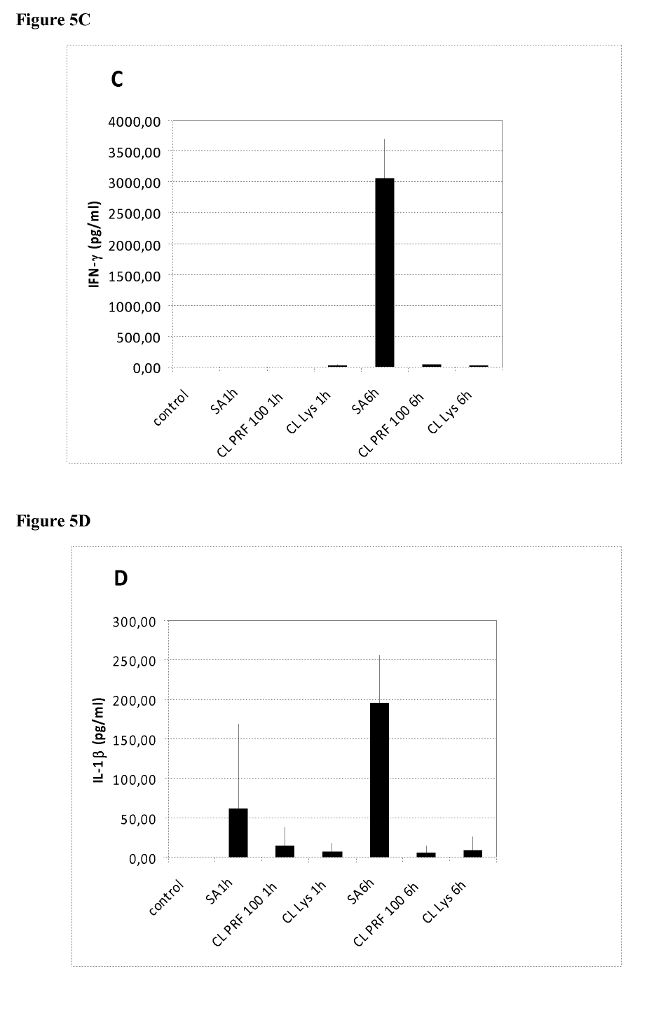

FIGS. 5A-D—A schematic representation of the time dependence of cytokine levels in animals after injection of S. aureus cells with or without pre-treatment of endolysin PRF-100 as depicted in SEQ ID NO:1 or Lysostaphin. Animals were infected with 6.5×10⁷ S. aureus cells or 6.5×10⁷ S. aureus cells preincubated for 3 hours at 37° C. in the presence of endolysin (PRF-100; 150 μg/ml) or Lysostaphin (150 μg/ml). Blood of animals were taken 1 hour or 6 hours after injection of S. aureus cells or lysine treated cells and serum was prepared. The mean values and standard deviation (n=3) of A, TNF-α concentrations; B, IL-6 concentrations; C, IFN-γ concentrations; D, IL-1β concentrations in sera of rats are blotted. Control: not treated animals; SA: S. aureus treated animals; CL PRF 100: Cell lysat of PRF 100 treated S. aureus cells; CL Lys: Cell lysat of Lysostaphin treated S. aureus cells.

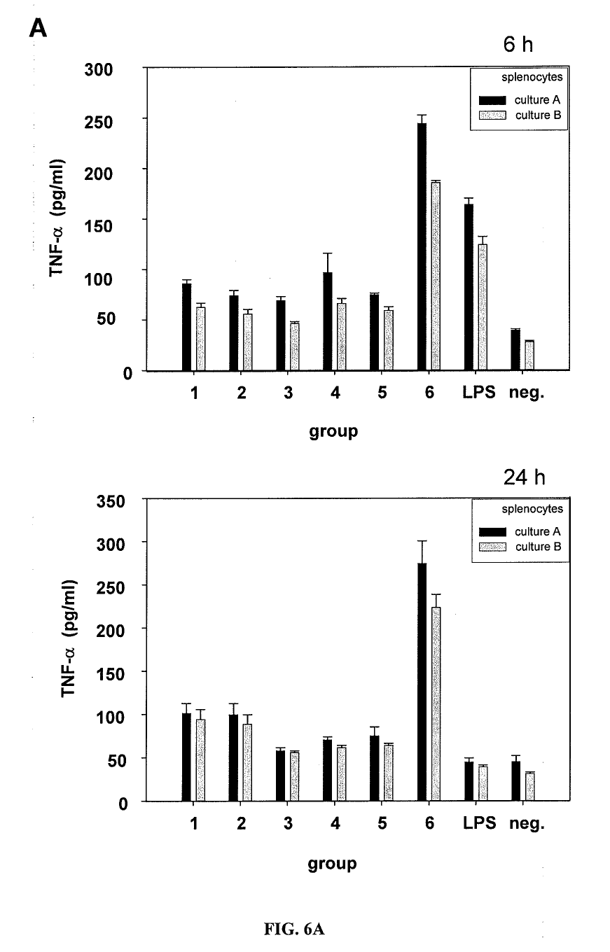

FIGS. 6A-B—Stimulatory properties of S. aureus cell wall preparations with or without pre-treatment of cell wall degrading enzymes in the murine splenocyte model system. Splenic cells of mice were incubated for 6 h or 24 h with 12.5 μg untreated cell wall preparations of S. aureus (group 6) or cell walls, preteated either with 5 μg/ml PRF 100 (group 1), 2.5 μg/ml Lysostaphin (group 2), 10 μg/ml Mutanolysin (group 3) or combinations of 5 μg/ml PRF 100 and 10 μg/ml Mutanolysin (group 4) or 2.5 μg/ml Lysostaphin and 10 μg/ml Mutanolysin (group 5). LPS of E. coli O111:B4 served as positive control for activation of cytokine response of primary murine splenocytes. Activation of the innate immune system was analysed by determining the secretion of proinflammatory cytokines TNF-a (A) and IL-6 (B). Experiments were performed in duplicate (culture A and culture B).

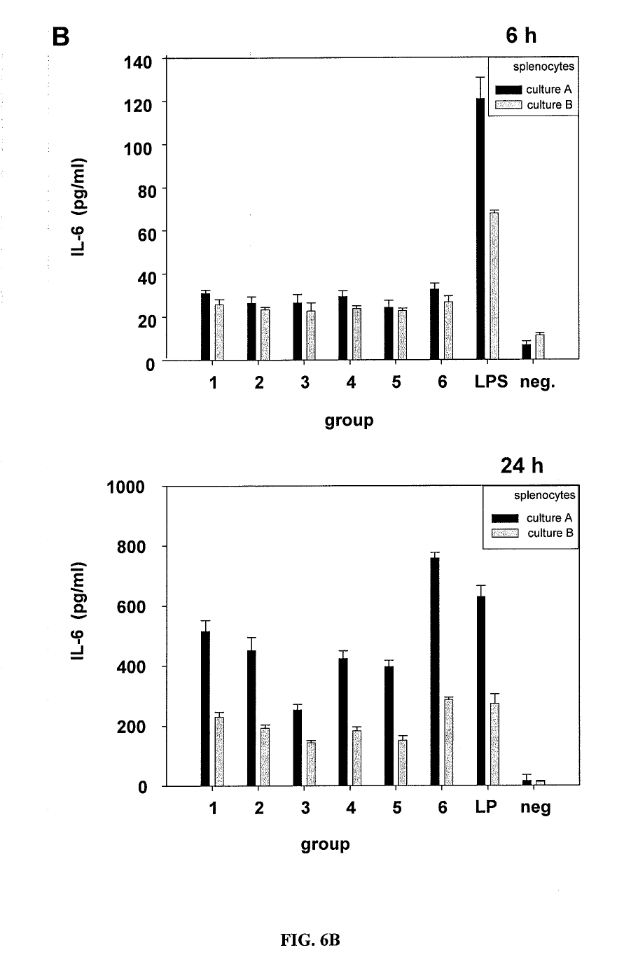

### DETAILED DESCRIPTION OF THE INVENTION

### A. Definitions

The term “bacterial cell wall degrading enzyme” as used herein refers to an enzyme which is suitable to hydrolyse bacterial cell walls. Said enzyme may be an endolysin, autolysin, lysostaphin, lytic enzyme similar to lysostaphin or a muralytic enzyme.

The term “endolysin” as used herein refers to an enzyme that comprises at least one of the following activities of which the “enzymatically active domains” (EADs) of the endolysins are constituted: endopeptidase, N-acetyl-nuramoyl-L-alanine-amidase (amidase), N-acetyl-muramidase or N-acetyl-glucosaminidase (lysozyme). Either, the enzyme is phage encoded or it is derived from related enzymes coded by bacteria, the so-called “autolysins”. In addition, the endolysins usually contain also regions which are enzymatically inactive, and bind to the cell wall of the host bacteria, the so-called CBDs (cell wall binding domains).

The term “domain” as used herein refers to a subunit of an endolysin which is ascribed a specific function and can also coincide with structural domains. The term domain is preferentially used to describe the antagonism between EAD which can be composed of more than one module and CBD domains.

The term “CBD” as used herein refers to the cell wall binding domain of an endolysin, which is often found at the C-terminus of the protein. CBD domains have no enzymatic activity in terms of hydrolyzing the cell wall, but often mediate binding of the endolysin to the bacterial cell wall. CBD may contain an SH3-domain.

The term “EAD” as used herein refers to the enzymatically active domain of an endolysin which is responsible for hydrolysis of the bacterial peptidoglycan. It contains at least one of the enzymatic activities of an endolysin. The EAD can also be composed of more than one enzymatically active module.

A “CHAP” domain (cysteine, histidine-dependent amidohydrolases/peptidases) is a region between 110 and 140 amino acids that is found in proteins from bacteria, bacteriophages, archaea and eukaryotes of the Trypanosomidae family. The proteins may function mainly in peptidoglycan hydrolysis. The CHAP domain is commonly associated with bacterial type SH3 domains and with several families of amidase domains. CHAP domain containing proteins may utilize a catalytic cysteine residue in a nucleophilic-attack mechanism. The CHAP domain contains two invariant amino acid residues, a cysteine and a histidine. These residues form part of the putative active site of CHAP domain containing proteins.

The term “ami” as used herein describes an enzymatically defined module which exhibits amidase activity, i.e. it hydrolyzes the amide bond between N-acetylmuramine in the peptidoglycan backbone and the adjacent amino acid which is usually L-ala in the peptide linker. The amidase are often metal ion dependent for activity.

The term “SH3” domain which is sometimes also called Src homology 3 domain as used herein describes a small non-catalytic protein domain of about 60 amino acids which is characteristic for proteins which interact with other binding partners. It is identified via a praline-rich consensus motif. The SH3 domain is usually located within the CBD.

The term “shuffling” as used herein refers to the combination of different fragments of polypeptides from different enzymes into new polypeptide constructs. In this context, the enzymes are preferentially endolysins, and the fragments are preferentially modules. Usually, the fragments are combined by molecular biological methods on nucleic acid level. Small linker sequences may be introduced between the fragments for structural or cloning reasons.

### B. Endolysin

Endolysins are enzymes, used by bacteriophages to facilitate the release of newly assembled bacteriophages at the end of the lytic life cycle. Endolysin enzyme activities may be divided into five classes: (1) N-acetylmuramidases (lysozymes), (2) endo-β-N-acetylglucosaminidases, and (3) lytic transglycosylases, which all cleave the sugar moiety of peptidoglycan, (4) endopeptidases, which cleave the peptide moiety, and (5) N-acetylmuramoyl-L-alanine amidases, which cut the amide bond between sugar backbone and peptide linkers. Endolysins show a modular organization exhibiting a combination of different polypeptide domains showing at least one enzymatic activity and a cell binding activity, the so-called EADs (enzymatically active domains) and CBDs (cell binding domains), respectively. Mostly, EADs are located at the N-terminal part of the endolysins, and CBDs at the C-terminal parts, but there are also exceptions of this rule of thumb. It is also shown that modules can be exchanged between different cell wall lytic enzymes producing new functional enzymes, which sometimes exhibit even new functional properties (Diaz et al., 1990, Proc. Natl. Acad. Sci. USA, 87, 8125-8129; Croux et al., 1993, Molecular Microbiology, 9, 1019-1025; Donovan et al., 2006, Appl. & Environm. Microbiol., 72, 2988-2996).

### C. Autolysin

Autolysins are a group of enzymes that exist in all bacteria containing peptidoglycan. The peptidoglycan matrix is very rigid, so these enzymes break down the peptidoglycan matrix in small sections so that growth and division of cells can occur. Autolysins do have similar or same enzymatic activities like endolysins. Autolysins are naturally produced by peptidoglycan containing bacteria, but excessive amounts will degrade the peptidoglycan matrix and cause the cell to burst due to osmotic pressure. Gram positive bacteria regulate autolysins with teichoic acid molecules attached to the tetrapeptide of the peptidoglycan matrix.

### D. Lysostaphin

Lysostaphin is an endopeptidase encoded by Staphylococcus simulans. to attack Staphylococcus aureus.

### E. Muralytic Enzymes

Muralytic enzymes are defined as enzymes cleaving the murein or petidogylcan.

### F. Mutanolysin

Mutanolysin is a N-acetylmuramidase encoded by Streptomyces globisporus.

### G. Sepsis

Bacterial products commonly leading to septic shock are lipopolysaccharide (LPS) or endotoxin, peptidoglycan, bacterial lipoproteins, mycobacterium lipoproteins and lipoarabinomannan, flagellin and heat shock proteins (SOM 208 Medical Microbiology Syllabus). Once the infectious agent or toxin activates the immune system, inflammation characterized by the usual symptoms (pain, swelling, redness and heat) follows.

The cell wall of gram-positive bacteria contains lipoteichoic acid (LTA) and peptidoglycan (PepG), which can activate leukocytes, stimulate the generation of proinflammatory cytokines, and hence, cause a moderate systemic inflammatory response syndrome. The endogenous vasodilator autacoid nitric oxide (NO) is generated by three different isoforms of NO synthase (NOS), two of which are expressed constitutively (eNOS in endothelium, nNOS in brain), the third (iNOS) induced by endotoxin (LPS) or cytokines. There is now substantial evidence that an enhanced formation of NO by iNOS contributes to the circulatory failure (hypotension, and vascular hyporeactivity to vasoconstrictors) and possibly the organ injury associated with endotoxemia. LTA is a macroamphiphile, equivalent to LPS in gramnegative bacteria, containing a substituted poly-glycero-phosphate backbone attached to a glycolipid. The glycolipid content in LTA resembles the bacterial membrane composition, which usually varies in a genus-specific manner. LTA from Staphylococcus aureus can cause a moderate induction of iNOS which (in murine macrophages) requires the activation of tyrosine kinases and NFk B. Although LTA from S. aureus can cause moderate hypotension in the rat, LTA (unlike S. aureus itself) does not cause multiple organ failure or death in this species. However, we have discovered recently that LTA and PepG act in synergy to release TNFalpha and IFNgamma, to induce iNOS, and to cause shock and multiple organ failure in anesthetized rats.

In addition, it is not known which of the structural components of PepG (or LTA) is essential for the observed synergism. PepG is a large polymer that provides stress resistance and shape-determining properties to bacterial cell walls. This polymer contains long sugar chains of two alternating sugar derivatives, N-acetylglucosamine (NAG) and N-acetylmuramic acid (NAM), which are highly cross-linked by peptide subunits and bridges. The peptide subunit (or stem peptide) consists of alternating 1- and d-amino acids, up to four or five in length, and is connected to the COOH group of NAM. Among different bacterial species, the structure of the sugar chains is highly preserved, while the composition of the peptide subunits varies. The present inventors now have investigated which of the structural elements of PepG are essential to synergize with LTA to cause the induction of iNOS in macrophages, and shock and multiple organ failure in vivo. The results of the investigation lead to the present invention.

Thus, one aspect of the present invention is directed to a method of treatment or prophylaxis of an inflammatory disease, in particular sepsis, in particular sepsis caused by gram-positive bacteria. The method comprises the steps of administering to a subject in need thereof a sufficient amount of bacterial cell wall degrading enzymes. Said subject may be a human subject or an animal, in particular animals used in livestock farming and/or dairy farming such as cattle.

In particular the bacterial cell wall degrading enzyme is a bacteriophage endolysin, an autolysin, lysostaphin, mutanolysin, lytic enzyme similar to lysostaphin and murolytic enzymes. Said bacterial cell wall degrading enzymes may be naturally occurring enzymes which have been biochemically isolated or which have been recombinantly generated. Said enzymes further may have been synthetically engineered, in particular by amino acid modifications like substitutions, deletions or additions. Said bacterial cell wall degrading enzymes combine the activities of binding to the bacterial cells, lysis of bacterial cells and neutralizing of cell wall degradation products.

In a preferred embodiment the endolysin is composed of a CBD domain of one endolysin and an EAD of another endolysin. In a particularly preferred embodiment said recombinant endolysin comprises an endolysin cell binding domain of the SH3 type. Preferably, the CBD domains are selected from the endolysin CBD domains of ply_USA, or ply_pitti20. As CBD of ply_USA in particular the sequence as denoted in SEQ ID NO:2 is preferred. As CBD of ply_pitti20 in particular the sequence as denoted in SEQ ID NO:3 is preferred.

In a particularly preferred embodiment said recombinant endolysin comprises an enzymatically active domain which derives from a different endolysin than the CBD. Illustrative examples for such recombinant endolysin variants and consists of the EAD from ply_pitt26 and the CBD from ply_USA.

In a further preferred embodiment the endolysin may comprise one or more of the following modifications: amino acid substitutions, deletions or additions. Particularly preferred is the polypeptide according to SEQ ID NO:1. Said polypeptide is derived from ply_pitti26, i.e. the CBD of ply_pitti 26 is replaced with the CDB of plyUSA as depicted in SEQ ID NO:2 and said polypeptide having the following five single amino acid substitutions L55H, L56T, E163Q, R167A and Y200H.

In particular said bacterial cell wall degrading enzymes may be administered in case the inflammatory disease, in particular a sepsis is caused by gram positive bacteria e.g. Staphylococcus, Streptococcus or Enterococcus.

During sepsis the cytokine cascade is strongly activated in response to bacterial stimuli. In the early phase the proinflammatory cytokines (e.g. IL-6 and TNFa) are upregulated which is triggered by toxins derived from cell wall components (Peptidoglycan=PG and Lipoteichoic acids=LTA). During ongoing sepsis further inflammatory pathways and NO production are started, ending up with multiple organ failure and death. During gram positive sepsis the main stimulator of the inflammatory response are the LTAs and PG-fragments of the bacterial cell walls. Thereby, LTA in combination of PG-fragments act synergistically and lead to a pronounced onset of sepsis.

It has been shown, that different cell wall degrading enzymes (PRF-100, Lysostaphin, Mutanolysin) exhibit different potency in eliminating bacteria from the blood stream. Additionally, the inventors found that such enzymes neutralize and/or degrade the cell wall toxins of bacteria and thereby suppress the pro-inflammatory cytokine response and subsequent cascades. Surprisingly, the efficiency of the enzymes to kill the bacteria is not correlated with their ability to suppress the proinflammatory response, in vivo and in vitro.

In other words, the bacterial cell wall degrading enzymes have the capability to neutralize the cell wall degradation products in a way that the release of proinflammatory cytokines is significantly reduced. Neutralization in this respect shall mean, the binding and thereby neutralizing of LTA, the binding and thereby neutralizing PG and its fragments, and further hydrolysis of PG and its fragments and thereby destroying the activating potential of PG.

Thus, said method of treatment may be for the treatment or prophylaxis of inhibiting or reducing the level of proinflammatory cytokines like TNF-a, IL-6, IL-1β, IFN-g. The reduction of the level of proinflammatory cytokines like TNF-a, IL-6, IL-1β, IFN-g is preferably about 40%, 50% or 60% and more preferably about 70%, 75%, 80%, 85%, 90% or 95%. The reduction of the level of said proinflammatory cytokines can be determined by methods well known by a person skilled in the art and as e.g. described in the examples of the present invention.

Furthermore, the neutralizing capability of bacterial cell wall degrading enzymes can be measured by reducing the stimulation of proinflammatory cytokines.

Furthermore, the method of treatment or prophylaxis comprises the steps of administering to a subject in need thereof a sufficient amount of bacterial cell wall degrading enzymes in combination with conventional antibacterial agents, such as antibiotics. Furthermore, said enzymes may be administered in combination with other endolysins, autolysins, lysostaphins, mutanolysins, lytic enzymes similar to lysostaphin or murolytic enzymes. Furthermore, said enzymes may be administered in combination with conventional antibacterial agents, such as antibiotics and other endolysins, autolysins, lysostaphins, mutanolysins, lytic enzymes similar to lysostaphin or murolytic enzymes.

The dosage and route of administration used in a method of treatment or prophylaxis according to the present invention depends on the specific inflammatory disease to be treated. The route of administration may be for example in particular embodiments parenteral, intravenous, rectal or any other route of administration.

For application of a bacterial cell wall degrading enzyme alone or in combination with other enzymes or conventional antibacterial agents to a site of infection (or site endangered to be infected) the enzymes may be formulated in such manner that the enzymes are protected from environmental influences such as proteases, oxidation, immune response etc.

In a further aspect of the present invention the above mentioned bacterial cell wall degrading enzymes are a component of a pharmaceutical composition, which optionally comprises a carrier substance.

### EXAMPLES

All cloning procedures were performed using standard techniques according to Sambrook et al. (Molecular cloning. A laboratory manual; 2nd ed. Cold Spring Harbor Laboratory Press 1989). Mutations and deletions were also introduced using standard techniques.

### Example 1

### Cultivation and Preparation of S. aureus Cells

An overnight culture of Staphylococcus aureus DMSZ 11823 was diluted into fresh BHI culture medium and grown at 37° C. to mid-log phase (OD600 nm=0.78). Then, cells were harvested by centrifugation and resuspended in equal volume of PBS buffer. To control the number of viable cells in this preparation, dilution series of the cell suspension were plated onto LB agar plates, grown over night at 30° C. and the number of colony forming units determined. Determination of cfus results in 6.5×107 cell/ml.

### Example 2

### Preparation of Cell Debris

1.2×10⁷ S. aureus cells were buffered in ½ TBS buffer and lysed with 150 μg/ml of an enzyme according to SEQ ID NO:1 (designated PRF-100) or 150 μg/ml Lysostaphin for 3 h at 37° C. To disrupt aggregates in this solution the debris was sonified for 30 seconds on ice, aliquoted and frozen at −80° C.

### Example 3

### Infection/Treatment of Rats with S. aureus/PRF-100

Narcotized 250 gram sprague-Dawley (CD) rats were infected with 1 ml of S. aureus solution. One minute later, S. aureus infected animals were treated with an enzyme according to SEQ ID NO:1 solution (16 mg/kg) or a corresponding volume of an enzyme storage buffer.

### Example 4

### Preparation of Blood, Serum Samples and Heart Tissue of the Animals

After 5, 60 and 360 minutes narcotized animals were killed, blood samples were collected and either heparinised or serum samples prepared. Serum samples of the animals were analysed for TNF-a and IL-6 levels corresponding to manufacturers instructions (Quantakine cytokine kits; R&D systems). Blood samples were analysed for the number of living S. aureus cells (cfu/ml) by plating dilution series of Proteinase K treated samples. Heart tissue of the animals, infected with living S. aureus and treated with an enzyme according to SEQ ID NO:1 or buffer, were prepared, homogenised in PBS buffer and number of living cells (cfu) were determined by plating dilution series onto LB agar plates.

### Example 5

### Infection/Pre-Treatment of Rats with S. aureus/PRF-100 or Lysostaphin

Narcotized 250 gram Sprague-Dawley (CD) rats were pretreated with 16 mg/kg of PRF-100 or 8 mg/kg of Lysostaphin, 15 minutes before infection with S. aureus cells (1×10⁷ cfu/ml). Ten minutes after infection blood was withdrawn, serum prepared and 100 μl plated onto LB agar plates. After growth over night at 30° C., the number of colonies forming units (cfu) were determined.

### Example 6

### Infection of Rats with S. aureus Cells and S. aureus Cells Pretreated with PRF-100 or Lysostaphin

Narcotized 250 gram Sprague-Dawley (CD) rats were infected with 1×10⁷ S. aureus cells or cell debris generated by PRF-100 or Lysostaphin. Blood of animals were taken 1 hour or 6 hours after injection of S. aureus cells or lysine treated cells and serum was prepared. Measurement of proinflammatory cytokines TNF-α, IL-6, IFN-γ and IL-1β was performed using commercial Quantikine® ELISA kits following the instructions of the manufacturer.

### Example 7

### Preparation of S. aureus Cell Walls

An 2 liter over night culture of S. aureus (DSMZ11823) was harvested by centrifugation at 4500×g and resuspended in 20 ml of buffer A (20 mM Tris pH 7.5; 250 mM NaCl and 1 mM MgCl2). Cells were mechanically disrupted using a Microfluidizer. After centrifugation the pellet was dissolved in buffer A and 0.1 mg/ml DNAse and RNAse (Biozyme Laboratories) were added. After 30 hours at 30° C., 1 mg/ml Trypsin (Sigma) was added and incubated for additional 6 hours at 37° C. At least, 0.1 mg/ml Proteinase K (Applichem) was added and incubated for further 24 hours. After the protease treatment the cell walls were washed twice with buffer A. To destroy the remaining protease activity the dissolved cell walls were heat treated for 1 h at 95° C. The optical density at 600 nm of the cell wall solution was adjusted to 100 with buffer A. 1.5 ml aliquots of the solution were lyophilized.

### Sample Preparation:

0.0710 g Lyophilized cell wall preparations of S. aureus (DSMZ11823) were solubilized in 960 μl H₂O (endotoxin-free) resulting in a solution with a final concentration of 0.05 g/ml. Cell wall solutions were diluted 1/100 in Y2 TBS buffer (OD 0.73). Treatment with cell wall enzymes was performed for 6 hours at 30° C.

### Example 8

### Generation of Splenic Cells of BALB/c Mice

BALB/c mice were sacrificed by cervical dislocation and spleens were removed aseptically. Then spleens were placed in 50 ml Falcon tubes including 10 ml splenocyte medium/spleen and kept on room temperature (not longer than 15 min). Medium was replaced by the same volume of fresh splenocyte medium and spleens were transferred onto a 70 μM cell strainer, which is placed on a 50 ml Falcon tube. Single cell suspensions were generated by grinding the spleen against the cell strainer with the plunger of a 5 ml syringe until mostly fibrous tissue remains left. Singularized cells were aspirated from the cell strainer by repeatedly adding 2 ml splenocyte medium. Obtained cell suspensions were sedimented by centrifugation at 300×g for 5 min at room temperature and pelleted cells were resuspended in 5 ml/spleen ACK hemolysis buffer by gently but thoroughly pipetting with a 10 mL or 25 mL plastic pipette. Then, cell suspension was centrifuged at 300×g for 5 min at room temperature and washed three times with 10 mL/spleen splenocyte medium. At each washing step the cell suspension was separated from aggregated fibrous tissue. Finally, cells were resuspended in 5 ml/spleen splenocyte medium, counted and adjusted to a final concentration of 2×10⁶ cells/ml in splenocyte medium and preserved at 37 C.° (not longer than 30 min) until use.

### Example 9

### Determination of Cytokine Levels from Mice Splenic Cells

Each 2×10⁶ freshly isolated splenic cells obtained from 3 BALB/C mice were transferred in 1 ml splenocyte medium in 24 well cell culture plates and stimulated with 12.5 μg/ml S. aureus cell wall preparation, pretreated either with 5 μg/ml PRF100 (sample 1), 2.5 μg/ml Lysostaphin (sample 2), 10 μg/ml Mutanolysin (sample 3), 5 μg/ml PRF100+10 μg/ml Mutanolysin (sample 4) or 2.5 μg/ml Lysostaphin+10 μg/ml Mutanolysin (Sample 5). Cells treated with untreated cell wall preparation (sample 6) or with 1 μg/ml LPS or 10 μl lyophilisation buffer served as positive and negative controls. All stimulations were performed in five independent stimulation batches. After an incubation of cells for 6 or 24 hours at 37° C. in a humidified incubator with 5% CO₂, cells/cell debris were removed from the cell culture supernatants by low speed centrifugation. Obtained cell-free culture supernatants were frozen at −80° C. until determination of cytokines. Measurement of proinflammatory cytokines IL-6 and TNF-α was performed using commercial BD OptEIA™ kits following the instructions of the manufacturer.

1. A method of treatment of an inflammatory disease in a human or animal subject, comprising administering an efficient amount of bacterial cell wall degrading enzymes to said subject.

2. The method according to claim 1, wherein the bacterial cell wall degrading enzyme is an endolysin, autolysin, lysostaphin, mytanolysin, lytic enzyme similar to lysostaphin or murolytic enzyme.

3. The method according to claim 1, wherein the bacterial cell wall degrading enzyme is administered in combination with conventional antibacterial agents, such as antibiotics.

4. The method according to claim 1, wherein the bacterial cell wall degrading enzyme is administered in combination with conventional antibacterial agents, such as antibiotics and other endolysins, mytanolysins, autolysins, lysostaphins, lytic enzymes similar to lysostaphin or murolytic enzymes.

5. A pharmaceutical composition comprising a bacterial cell wall degrading enzyme.

6. A pharmaceutical composition according to claim 5, wherein the bacterial cell wall degrading enzyme is an endolysin, autolysin, lysostaphin, mytanolysin, lytic enzyme similar to lysostaphin or murolytic enzyme.

7. A pharmaceutical composition according to claim 5, further comprising a conventional antibacterial agent, such as an antibiotic agent.

8. A pharmaceutical composition according to claim 5, comprising different bacterial cell wall degrading enzymes.

9. A pharmaceutical composition according to claim 5, further comprising conventional antibacterial agents, such as antibiotics and other endolysins, autolysins, lysostaphins, mytanolysins, lytic enzymes similar to lysostaphin or murolytic enzymes.

10. A pharmaceutical composition according to claim 5, further comprising a pharmaceutical acceptable a carrier.
